(19) United States
(12) Patent Application Publication
(10) Pub. No.: US 2002/0137676 A1
(43) Pub. Date: Sep. 26, 2002

(54) Selective treatment of endothelial somatostatin receptors

(76) Inventors: York Hsiang, Vancouver (CA); Alison Buchan, Vancouver (CA); Julia G. Levy, Vancouver (CA); Philippe Maria Clotaire Margaron, Burnaby (CA)

Correspondence Address: MORRISON & FOERSTER LLP, 3811 VALLEY CENTRE DRIVE, SUITE 500, SAN DIEGO, CA, 92130-2332, US

(21) Appl. No.: 09/797,779

(22) Filed: Mar. 1, 2001

Related U.S. Application Data
(63) Continuation-in-part of application No. PCT/CA99/008800, filed on Sep. 1, 1999.

(30) Foreign Application Priority Data
Sep. 1, 1998 (CA) 2,246,791

(51) Int. Cl. A61K 38/31

(52) U.S. Cl. 514/12

(57) ABSTRACT

The invention provides for the use of somatostatin receptor selective ligands (selective for SSTR1 or SSTR4) to treat human endothelial cells and to formulate medicaments for human use. The medicaments may for example be used to treat an angiogenic disease. In various embodiments, the angiogenic disease may for example be macular degeneration or a solid tumor. The SSTR1 or SSTR4 selective agonists may include the SSTR1 agonist (des-AA^{1,2,5} [DTrp⁸,IAamp⁹]SS).

### RELATED APPLICATION

This application is a continuation-in-part of PCT Application No. PCT/CA99/00800, filed Sep. 1, 1999 and which designates the United States, which claims benefit of priority to Canadian patent application serial no. 2,246,791, filed Sep. 1, 1998, both of which are hereby incorporated by reference as if fully set forth.

### FIELD OF THE INVENTION

The invention is in the field of therapeutic uses for selective peptide and nonpeptide somatostatin receptor ligands.

### BACKGROUND OF THE INVENTION

Somatostatin (SS) is an endogenous cyclic peptide found in two major native molecular forms of 28 and 14 amino acids (SS28 and SS14 respectively, SS was initially described as a somadomedin release-inhibiting factor, and is consequently still called SRIF in some of the literature). SS has disparate, but primarily inhibitory, roles in a variety of physiological systems, either acting directly on cellular functions or as an antagonist of stimulatory factors (Coy et al. 1993, J. Pediatric Endocrinol. 6:205). The multiplicity of effects of SS on physiological processes reflects both its widespread distribution in vivo, and the existence of multiple SS receptor subtypes.

The effects of SS are transduced by a family of SS receptors (SSTRs), of which 5 (SSTR1 through SSTR5) have been cloned (Coy et al. 1993, supra). These receptors may be divided into two sub-groups on the basis of their relative sequence similarities and affinity for SS analogues (Hoyer et al., 1995, Trends Pharmacol Sci 16:86). One sub-group consists of SSTR2, SSTR3 and SSTR5. The second sub-group comprising SSTR1 and SSTR4. The physiology of the first sub-group of receptors has been more thoroughly characterized, due in part to the relative availability of SS analogues that are selective for these SSTRs, particularly SSTR2. It is however known that all 5 SSTRs share some mechanistic features, for example all 5 have been shown to be coupled to G-proteins and to regulate intracellular cAMP levels, in part, through activation of Gᵢ (Patel et al. 1994, Biochem. Biophys Res. Commun. 198:605).

SS has an extremely short half life in vivo, rendering it unsuitable for most therapeutic uses. For therapeutic applications, a variety of short peptide analogues of SS have been identified, particularly agonists of the first sub-group of SSTRs (see for example U.S. Pat. Nos. 4,485,101 issued Nov. 27, 1984; 4,904,642 issued Feb. 27, 1990; 5,147,859 issued Sep. 15, 1992; 5,409,894 issued Apr. 25, 1995; 5,597,894 issued Jan. 28, 1997; and, International Patent Publications: WO 97/01579 of Jan. 16, 1997 and WO 97/47317 of Dec. 18, 1997; all of which are hereby incorporated by reference).

Among the most thoroughly characterized of the peptide SSTR agonists are octreotide (Sandoz Ltd., Basel, Switzerland) and angiopeptin (sometimes referred to as BIM 23014). Octreotide is recognized as an SSTR2 selective agonist (Yang et al., 1998, PNAS USA 95:10836). Angiopeptin is recognized as an SSTR2/SSTR5 selective agonist (Alderton et al., 1998, Br. J. Pharmacol 124(2):323). U.S. Pat. No. 5,750,499 (issued May 12, 1998 to Hoeger et al., incorporated herein by reference) discloses what are claimed therein to be the first SSTR1 selective agonists (also described in Liapakis et al., 1996, The J. of Pharmacology and Experimental Therapeutics 276(3)1089, incorporated herein by reference), one of which is identified as des-AA^{1,2,5} [DTrp⁸,IAamp⁹]SS (i.e. des-amino acid ^{1,2,5}[DTryptophan⁸, N-ρ-isoproply-4-aminomethyl-L-phenylalanine⁹]SS, abbreviated herein as the “SSTR1 '499 agonist”).

A number of nonpeptide somatostatin receptor subtype-selective agonists have been identified using combinatorial chemistry (Rohrer et al. 1998, Science 282:737, incorporated herein by reference). Included amongst the agonists identified by Rhorer et al., supra, are agonists selective for SSTR1 and SSTR4. Rhorer et al., supra, also disclose the apparent inhibition constant (K₁) for SS14 binding to the SSTR receptors, as shown in Table 1, and disclose methods of calculating that constant for SSTR selective ligands. Rhorer et al., supra, indicate that the SSTR1 and SSTR4 agonists disclosed therein were not physiologically active, in that they did not inhibit the release of growth hormone, glucagon or insulin in a model system. In contrast, a SSTR2 agonist is disclosed as having potent inhibitory effects on secretion of growth hormone, glucagon and insulin.

TABLE 1
SS14 SSTR Specificity (Kᵢ in nanomoles)*:

| | SSTR1 | SSTR2 | SSTR3 | SSTR4 | SSTR5 |
|---|---|---|---|---|---|
| SS14 | 0.4 | 0.04 | 0.7 | 1.7 | 2.3 |

*From Rohrer et al. 1998, Science 282:737.

It has been suggested that particular SSTR agonists may be useful in the treatment of a variety of diseases, particularly in light of favourable results of treatment in some animal models. For example, on the basis of the chicken chorioallantoic membrane (CAM) model, it has been suggested that SSTR2 agonists in particular may be effective inhibitors of angiogenesis (Woltering et al. 1997, Investigational New Drugs 15:77, in which SSTR2 binding activity of a number of agonists is correlated with the compounds anti-angiogenic activity). With respect to angiogenesis, SS itself has recently been shown to control growth of a xenografted Kaposi's sarcoma tumor in a nude mouse model, through inhibition of murine angiogenesis (Albini et al. 1999, The FASEB J. 13(6):647, wherein results are presented indicating that human endothelial cells express SSTR3). There is also abundant evidence that SSTR2 agonists, particularly angiopeptin, are effective in inhibiting intimal hyperplasia after arterial injury in animal models (Lundergan et al. 1989, Atherosclerosis 80:49; Foegh et al., 1989, Atherosclerosis 78:229; Conte et al., 1989, Transpl Proc 21:3686; Vargas et al., 1989, Transplant Proc 21:3702; Hong et al., 1993, Circulation 88:229; Leszczynski et al., 1993, Regulatory peptides 43:131; Mooradian et al., 1995, J. Cardiovasc Pharm 25:611; Light et al., 1993, Am J Physiol 265:H1265). It has been suggested that this therapeutic activity in animal models reflects the ability of angiopeptin to inhibit the release of growth factors from injured endothelial cells (Hayry et al., 1996, Metabolism 45(8 Suppl 1):101). In clinical studies, however, the use of the SSTR2/SSTR5 agonist angiopeptin to inhibit intimal hyperplasia causing restenosis in human patients has been inconclusive (Eriksen et al., 1995, Am Heart J. 130:1; Emanuelsson et al., 1995, Circulation 91:1689; Kent et al., 1993, Circulation 88:1506). The poor clinical efficacy of angiopeptin in clinical trials for the prophylaxis of restenosis following coronary angioplasty, in contrast to encouraging data from animal studies, has been attributed to a low intrinsic activity of angiopeptin at the SSTR2 receptor, combined with lack of agonist activity at the SSTR5 receptor (Alderton et al. 1998, Br. J. Pharmacol 124(2):323). SSTR2 agonists have also been found to be generally ineffective in the treatment of diabetic retinopathy (Kirkegaard et al., 1990, Acta Endocrinologica (Copenh) 122:766), despite the indications from in vitro and animal studies that such compounds exhibit anti-angiogenic activity.

Endothelial cells form a single cell layer lining all blood vessels in the human body, surrounded by other cell types such fibroblasts and smooth muscle cells. Endothelial cells are restricted to blood vessels. Endothelial-cell-mediated proliferative diseases such as angiogenic diseases and intimal hyperplasia continue to pose a significant health problem, caused by imbalances in the physiological system that regulates vascular remodelling. For example, ocular neovascularization in diseases such as age-related macular degeneration and diabetic retinopathy constitute one of the most common causes of blindness. Intimal hyperplasia causing restenosis or narrowing of the artery has been found to occur in 30-50% of coronary angioplasties and following approximately 20% of bypass procedures (McBride et al., 1988, N. Engl. J. Med. 318:1734; Clowes, 1986, J. Vasc. Surg. 3:381). Angiogenesis induced by solid tumor growth may lead not only to enlargement of the primary tumor, but also to metastasis via the new vessels.

### SUMMARY OF THE INVENTION

The inventors have made the surprising discovery that SSTR1 and SSTR4 are expressed on human endothlial cells, in vitro and in vivo, which contrasts with the presence of other SSTRs, particularly SSTR2, on endothelial cells in other animals. In addition, an SSTR1 binding ligand is shown to inhibit angiogenesis in a model system. Accordingly, the invention provides for the use of SSTR1 and SSTR4 ligands, including selective ligands such as, to treat human diseases. Agonist ligands are contemplated as advantageous in, but not limited to, diseases involving pathological neovascularization (angiogenesis). Antagonist ligands are contemplated as advantageous in, but not limited to, conditions requiring the activation of neovascularization (angiogenesis) or competition with SSTR1/SSTR4 mediated stomatostatin activity. In various embodiments, the angiogenic disease may for example be age-related macular degeneration, or a solid tumour. A SSTR1 selective ligand for use in the present invention may for example be the SSTR1 '499 agonist (des-AA^{1,2,5} [DTrp⁸,IAamp⁹]SS). In methods of treatment, therapeutically effective amounts of SSTR1 or SSTR4 ligands may be administered to a patient.

### BRIEF DESCRIPTION OF THE DRAWINGS

FIG. 1 is a graph showing the anti-angiogenic effects of SS14 in the ECV304/Matrigel model (Hughes, 1996, Experimental Cell Research 225:171-185), as disclosed in Example 1 herein.

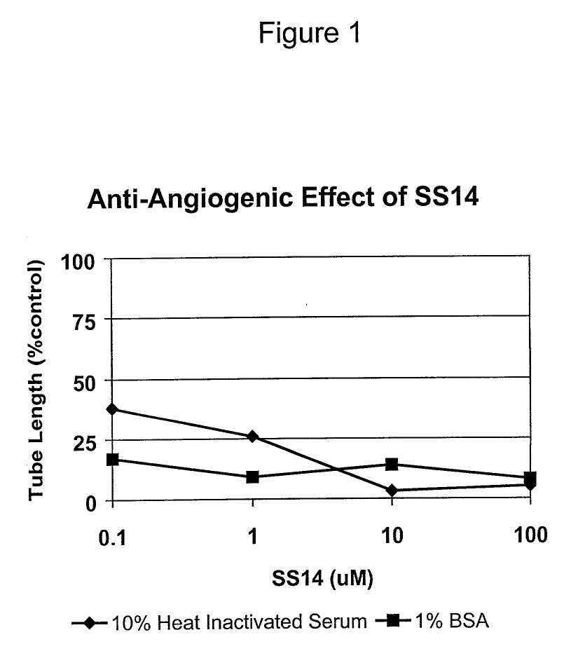

### DETAILED DESCRIPTION OF THE INVENTION

In one aspect, the invention provides therapeutic uses of SSTR1 and SSTR4 ligands. In some embodiments, the invention involves the use of SSTR1 and/or SSTR4 agonist ligands for the treatment of angiogenic diseases. Angiogenic diseases are characterised by pathological neovascularization as a result of inappropriate or unregulated angiogenesis, such as macular degeneration and solid tumour vascularization.

Diseases treated in accordance with various aspects of the invention may for example include proliferative retinopathies, such as retinopathy of prematurity, corneal graft rejection, retrolental fibroplasia, rubeosis, hypoxia, angiogenesis in the eye associated with infection; angiogenic aspects of skin diseases such as psoriasis; blood vessel diseases such as hemagiomas, and capillary proliferation within atherosclerotic plaques neovascularization; Osler-Webber Syndrome; myocardial angiogenesis; plaque neovascularization; telangiectasia; hemophiliac joints'; angiofibroma; and wound granulation. Diseases associated with ocular neovascularization treated with the invention include, but are not limited to, neovascularization of the choroid and retina (e.g.age-related macular degeneration, pathologic myopia, ocular histoplasmosis syndrome, diabetic retinopathy, diabetic macular edema), iris (e.g. neovascular glaucoma) cornea, and other abnormal neovascularization conditions of the eye. The use of the invention may also follow photodynamic therapy treatment for neovascularization conditions.

Other aspects include the treatment of diseases characterized by excessive or abnormal stimulation of endothelial cells, including but not limited to intestinal adhesions, Crohn's disease, atherosclerosis, scleroderma, and hypertrophic scars, i.e. keloids. SSTR1 and SSTR4 agonist ligands may also be useful in the treatment of diseases that have angiogenesis as a pathologic consequence such as cat scratch disease (Rochele ninalia quintosa) and ulcers (Helicobacter pylori). While the invention also includes use thereof in the treatment of angiogenesis associated with vascular injury or vascular surgical operation (fibroproliferative vasculopathy), it will be appreciated that in some aspects, the invention involves treatment of patients to inhibit angiogenesis where the patient has not undergone vascular injury or a surgical operation. Examples of procedures or conditions resulting in fibroproliferative vasculopathy include, but are not limited to, coronary bypass surgery, balloon angioplasty, PTCA (percutaneous transluminal coronary angioplasty), vascular allograft (leading to chronic allograft rejection and/or allograft arteriosclerosis), and diabetic angiopathy.

An alternative aspect of the invention comprises SSTR1 and SSTR4 agonist ligand treatments for cancers susceptible to anti-angiogenic treatment, including both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). In some aspects of the invention, SSTR1 and SSTR4 agonist ligands may also be useful in treating solid tumors arising from hematopoietic malignancies such as leukemias (i.e. chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, SSTR1 and SSTR4 agonist ligands may be useful in the prevention of metastases from the tumors described above either when used alone or in combination with radiotherapy and/or other chemotherapeutic agents.

Use of the present invention to treat or prevent a disease condition as disclosed herein, including prevention of further disease progression, may be conducted in subjects diagnosed or otherwise determined to be afflicted or at risk of developing the condition.

The invention may be practiced with any ligand that binds SSTR1 and/or SSTR4 with sufficient affinity to activate the receptors. Preferably, the ligand binds SSTR1 and/or SSTR4 with greater affinity than any other somatostatin receptor under the same conditions. In several aspects, the present invention relates to somatostatin receptor ligands that are selective for one or more of the somatostatin receptor subtypes. In this context, receptor-ligand binding assays may be carried out to determine the relative affinity of a compound for one or more of the somatostatin receptors, as for example described by Rhorer et al., 1998, Science 282:737. Of course the ligand may be obtained from any source, including isolation or purification from naturally occurring sources or synthetic production such as combinatorial chemistry. Naturally occurring ligands include proteins which may also be recombinantly produced after isolation of the nucleic acids encoding them. Standard molecular biology procedures and protocols may be used to conduct such an isolation.

In some embodiments, a compound will be ‘selective’ for a receptor if the apparent inhibition constant of the compound with respect to that receptor (Kᵢ, calculated as described by Rhorer et al., supra) is less than the Kᵢ of the compound with respect to another SS receptor, and in some embodiments at least ten fold less. In some embodiments, the selectivity of the ligands used in the invention may be greater than ten fold, such as 100 fold or 1000 fold. In some embodiments, the present invention encompasses compounds that are selective for more than one SSTR.

In general, various screening methods may be used to select ligands for alternative aspects of the invention, such as screening of combinatorial libraries (see Rhorer et al., supra). SSTR ligands that are identified by such screening methods may be assayed using SSTR-expressing cells, such as Chinese hamster ovary cells (CHO) K1, Chinese hamster lung fibroblast cells (CCL39), COS-1 or COS-7 cells, which may for example be used to express cloned human SSTR receptors. After their identification, SSTR ligands can be further screened to determine their activities as an agonist or antagonist for use in the present invention.

SSTR expressing cells may be produced by methods such as those described by Yamada et al. Proc.. Natl. Acad. Sci. U.S.A. 1992, 89:251-255; Rohrer et al.. Proc. Natl. Acad. Sci. U.S.A. 1993, 90: 4196; Siehler et al. Naunyn Schmiedbergs Arch. Pharmacol. 1999, 360(5): 488-499. SSTR1 and/or SSTR4 receptor gene sequences may be stably expressed in cell lines by various recombinant methods, such as the method of Yang et al. Proc.. Natl. Acad. Sci. U.S.A. 1998, 95(18):10836-10841, which uses CHO-K1 cells (American Type Culture Collection) which may be grown in 10% fetal calf serum to express stably transfected DNA encoding an SSTR such as SSTR1 or SSTR4.

In some aspects, the invention may utilize SSTR receptor ligand-binding assays, an exemplary protocol for which is briefly described as follows (Rhorer et al., supra; Rhorer et al., supra). The binding-assay mixture may include one or more specific receptors, such as SSTR1 or SSTR4, and a labelled reference ligand, for example 0.1 nm (final concentration) of the ligand 3-[¹²⁵] iodotyrosyl²⁵-somatostatin-28(leu⁸, O-Trp²², Tyr²⁵) (Amersham) in buffer (such as 50 mM tris-Hcl, pH 7.8, 1 mM EGTA, 5 MM Mg₂Cl₂, 10 ug/ml leupeptin, 10 μg/ml pepstatin, 200 μg/ml bacictracin and 0.5 ug/ml aprotinin) and 0.01 to 10,000 nM range of the ligand to be tested. Somatostatin-14 (SS-14) may be used as the control. The assay may for example be performed in a 96 well polypropylene plates with a final volume of 200 μl per well, as follows. A 20 μl aliquot of the labelled somatostatin is added to each well of the plate, followed by 20 μl of the potential ligand and 160 μl of a CHO-K1 cell membrane SSTR receptor suspension. The assay is carried at room temperature for 45 min, after which time the the receptors are harvested onto 96-well glass fiber filter plates (Packard Unifilter GF/C) pretreated with 0.1 % polyethyleneimine. The plates are washed with cold 50 mM tris-Hcl (pH 7.8) and dried overnight. The radioactivity of each sample is measured in a scintillation counter. The results are expressed as Ki (nM) values and compared with the control. Alternatively, a competitive inhibition method such as the method described by Siehler et al. Naunyn Schmiedbergs Arch. Pharmacol. 1999, 360(5): 510-521, may also be used.

In some embodiments, SSTR1 or SSTR4 ligands may for example be selected from: the multi-tyrosinated somatostatin analogs disclosed in U.S. Pat. No. 5,597,894, issued Jan. 28, 1997; cyclic peptides disclosed in U.S. Pat. No. 6,001,960, issued on Dec. 14, 1999; DOTA-(D)betaNal1-lanreotide (DOTALAN) and other analogs described by Smith-Jones et al. (1999, Endocrinology 140 (11):5136-48); chimeric peptides (Liapakis et al. 1996, Metabolism 45 (8 Supp 1):12-13; Siehler et al. Naunyn Schmiedbergs Arch. Pharmacol. 1999, 360(5): 500-509); nonpeptide somatostatin agonists (Liu et al. Curr Pharm Des April 1999;5(4):255-63); and L-362855 (Smalley et al., 1998, Br J Pharmacol 125(4):833-41).

In one aspect, the present invention utilises an established model system for assaying the effect of SSTR ligands on human angiogenesis. In one embodiment, the model system comprises the spontaneously transformed human umbilical vein endothelial cell line, ECV304, grown on a Matrigel substrate (Hughes, 1996, Experimental Cell Research 225:171-185). Matrigel is a solubilized basement membrane extract that promotes the differentiation of endothelial cells into capillary tube-like structures in vitro. It has been shown that cytoskeletal reorganization occurs when human umbilical vein endothelial cells undergo the morphological changes associated with neovascular tube formation on a Matrigel substrate (Grant et al., 1991, In Vitro Cell Dev. Biol. 27A(4):327-36.). As disclosed in Example 1 herein, using the in vitro angiogenesis model comprising ECV304 cells on a Matrigel substrate, it has been shown in the context of the present invention that SS14 inhibits angiogenesis. At sub-micromolar and higher concentrations, SS14 was found to significantly inhibit neovascular growth in this model system. These results indicate that SS14, which is an agonist of all somatostatin receptor subtypes (see Table 1), acts on human endothelial cells as an angiogenesis inhibitor. This assay can also be used to identify antagonist ligands that stimulate the process of angiogenesis.

The present inventors have further demonstrated that the ECV304 cells only express the SSTR1 and SSTR4 receptor subtypes, and do not express SSTR2, SSTR3 or SSTR5 mRNA in quantities detectable by RT-PCR (see Example 2 herein). Accordingly, the demonstrated anti-angiogenic effects of SS14 on ECV304 cells must be mediated by SSTR1 and/or SSTR4. The present inventors have also demonstrated that an SSTR1 selective ligand agonist has similar physiological effects on ECV304 cells as does SS14, particularly disassembly of actin stress fibres and formation of lamellipodia (see Example 3 herein). In alternative embodiments of the invention, SSTR1 and SSTR4 agonist ligands may be used to have anti-angiogenic effects on human endothelial cells, just as SS14 has an anti-angiogenic effect in the ECV304/Matrigel model system.

Somatostatin analogues have been shown to have therapeutic effects in a variety of animal models of proliferative disease, including angiogenesis and intimal hyperplasia. SSTR2 agonists in particular have been shown to be successful in ameliorating the pathologies of endothelial-cell-mediated proliferative disease models, such as CAM, arterial balloon injury in several animal species, and murine angiogenesis in a cancer model. The present inventors have determined that in contrast to animal models in which endothelial cells express SSTR2 (see Example 4 herein and Chen et al., 1997, J of Investigative Surgery 10:17), human endothelial cells and tissues express SSTR1 and SSTR4. This indicates that, whereas SSTR2 agonists are effective in treating animal models of human endothelial-cell-mediated proliferative pathologies or disease (see citations in Background), SSTR1 and SSTR4 selective agonists may be used to treat human patients in accordance with the present invention.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and are not limiting of the broad aspects of the invention as disclosed herein.

### EXAMPLE 1: Anti-Angiogenic Effect of SS14

This example shows the anti-angiogenic effect of SS14 on endothelial cell capillary-like tube formation in vitro, using an established model of angiogenesis. The model is based on the propensity of human endothelial cells, particularly ECV304 cells, to form capillary-like tubes on Matrigel, a basement membrane extract (Hughes, 1996, Experimental Cell Research 225:171).

Five mg vials of SS14 (Biomeasure Incorporated) were reconstituted using 1.0 mL 0.01% BSA/0.01N acetic acid/PBS to achieve a working stock of 3 mM. The human endothelial cell line ECV304 (ATCC) was cultured in Medium 199 (Ml 99, Sigma) supplemented with 2 mM L-glutamine (Gibco BRL), 1 mM sodium pyruvate (Gibco BRL), 5×10⁻⁵ M 2-mercaptoethanol (Sigma), 100 U/mL penicillin (Gibco BRL), 100 μg/mL streptomycin (Gibco BRL), 20 mM HEPES (Sigma), and optionally 10% heat-inactivated fetal calf serum (Gibco BRL) or 1% BSA. Cells were passed at a rate of 1:5 using 0.05% trypsin/0.005% EDTA (Gibco BRL) upon reaching confluence.

ECV304 cells (3.5×10⁴ in 0.5 mL complete M199 medium) were placed onto 24-well plates that were pre-coated with 0.125 mL of Matrigel (Becton-Dickinson). SS14 was immediately added to the ECV304 cells and the cells were incubated at 37° C. in a CO₂ humidified chamber. After 24 hours, images of tube-formation were recorded on film. Images were converted into a digital format using a Hewlett-Packard ScanJet 4C/T scanner, the summed length of capillary-like tubes was quantified using Optimas 6.1 image analysis software (Optimas Corp.).

FIG. 2 illustrates in graphic form the finding that SS14 inhibits neovascular tube formation in a dose-dependent manner. The graph in FIG. 2 shows that the inhibition of angiogenesis by SS14 was greater than 50% at all SS14 concentrations ranging from 0.1 μM to 100 μM, as measured by neovascular tube length relative to control samples that were not treated with SS14.

### EXAMPLE 2: Characterization of Human Endothelial Cells

The endothelial characterization of the ECV304 cells used in the present invention was confirmed by the detection of von Willebrand Factor (vWF) mRNA by RT-PCR and the detection of vWF by immunocytochemistry (vWF is a well known functional marker of endothelial cells that is involved in vivo in the blood clotting cascade). The ECV304 cells used herein also expressed the endothelial marker endothelial nitric oxide synthase (eNOS).

RT-PCR provided evidence for the presence of SSTR1 and SSTR4 mRNA in ECV304 cells and in a primary endothelial HUVEC cell line from umbilical veins. Neither cell lines expressed SSTR2, SSTR3 or SSTR5 mRNA, with the exception that later passages of some HUVEC cultures showed low levels of SSTR2.

The ECV304 and HUVEC endothelial cell lines were immunostained for SSTR1 and vWF, identifying the location of the SS receptors. The EC304 and HUVEC cell lines showed SSTR1 immunostaining in both the cytoplasm and on the plasma membrane. Localization of vWF in ECV304 cells and early passages of HUVEC cells showed that 95-100% of the cells were immunoreactive, however fewer cells were immunostained in the later passage of HUVECs (<60%).

In the present Example, ECV304 cells (American Type Culture Collection, Manassas, Va.) were cultured in Medium 199 (Sigma Chemical Co., St. Louis, Mo.) supplemented with 2 mM Glutamine, 24 mM sodium bicarbonate, 10 mM Hepes, penicillin (100 U/ml), streptomycin (0.1 mg/ml), and heat inactivated fetal calf serum (10%). HUVEC and AoSMC cells were obtained from Clonetics Corporation (Walkersville, Md.) with the required culture medium. The cell lines were grown in 75 cm2 Falcon flasks (Becton Dickinson Labware, Franklin Lakes, N.J.) for collection of RNA or seeded onto APES (Sigma) coated 20mm coverslips in 24 well Costar plates (Corning Inc., Coming, N.Y.) for histological studies. The following ECV304 cell line information is provided by the ATCC:

ATCC Number: CRL-1998, originally deposited in May 1992

Organism: Homo sapiens (human)

Designations: ECV304

Tissue: normal; umbilical vein; endothelium; endothelial

Morphology: cobblestone

Depositors: K. Takahashi

VirusSuscept: Semliki Forest virus (SFV)

Tumorigenic: yes, in BALB/c nu/nu mice

Karyotype: modal number=80

Products: angiotensin converting enzyme (ACE)

FluidRenewal: 2 to 3 times weekly

SubCulturing: Remove medium, add fresh 0.25% trypsin, 0.03% EDTA solution, rinse and remove trypsin. Allow the flask to sit at room temperature (or incubate at 37C) until the cells detach (usually 5 to 10 minutes). Add fresh medium, aspirate and dispense into new flasks.

SplitRatio: A ratio of 1:6 to 1:10 is recommended

Growth Properties: monolayer

Comments: ECV304 is a spontaneously transformed immortal endothelial cell line established from the vein of an apparently normal human umbilical cord (donor number 304). The cells are characterized by a cobblestone monolayer growth pattern, high proliferation potential without any specific growth factor requirement, and anchorage dependency with contact inhibition. Endothelium specific Weibel-Palade bodies were identified in electron microscopic studies. Immunocytochemical staining for lectin Ulex europaeus I (UEA-I) and PHM5 (anti-human endothelium as well as glomerular epithelium monoclonal antibody) was positive. The cells are negative for Factor VIII related antigen, for alkaline and acid phosphatases and for epithelial keratins. The cells will form tumors in BALB/c nu/nu mice, and will cause neovascularization on rabbit corneas. They are reported to produce pro-urokinase type PA (pro-u-PA) and express small amounts of intercellular adhesion molecule (ICAM-1), lymphocyte function associated antigen-3 (LFA-3). Vascular cell adhesion molecule (VCAM-1) and granular membrane protein-140 (GMP-140). Interleukin-1 (IL-1) and interferon exert suppressive effects on ECV304 cells. These cells also produce IL-6 after stimulation with IL-1. The line was cured of mycoplasma contamination by a 21 day treatment with BM Cycline. Further information may be included in the following references, which are hereby incorporated by reference: Takahashi et al., 1990, In Vitro Cell. Dev. Biol. 26:265; Takahashi and Sawasaki, 1991, In Vitro Cell. Dev. Biol. 27A:766; Takahasi and Sawasaki, 1992, In Vitro Cell. Dev. Biol. 28A:380). Propagation of the cell line may be carried out in ATCC Medium 199, 90%; heat-inactivated fetal bovine serum, 10%.

In the present Example, total RNA was isolated according to manufacturer's directions from tissue samples and cell lines lysed in Trizol solution (Gibco Life Technologies, Grand Island, N.Y.). Any DNA present was removed by incubation in the first strand buffer (25 mM Tris-HCl pH 8.3, 37.5 mM KCL, 1.5 mM MgCL₂ and 10 mM DTT) containing 1 mM dNTPs (Pharmacia), 10 U Rnasin (Pharmacia), and 2U of Dnase (Promega Corporation, Madison, Wis.) and heated to 37° C. for 30 min. The DNase was inactivated by heating to 75° C. for 5 min. A sample was removed and used as a PCR template to verify the absence of genomic DNA. The cDNA was synthesized from purified RNA using Superscript II reverse transcriptase (100 U MMLV, Gibco Life Technologies, Grand Island, N.Y.) according to the manufacturer's directions with oligo-dT primer ((Gibco), 10 U Rnasin (Pharmacia), and 1 mM dNTPs (Pharmacia)). Samples were incubated at 42° C. for 1 hour. The enzyme was inactivated by heating the samples to 75° C. for 15 min. The cDNA samples were stored at −20° C. prior to PCR.

For detection of SSTR subtypes in endothelial cell lines (and human blood vessels), oligonucleotide primers were synthesized on an Applied Biosystems Model 391 DNA synthesizer, as follows:

TABLE 2
HUMAN SSTR PRIMERS

| | | | PCR | |
|---|---|---|---|---|
| Primer | | Position in | product | Annealing |
| specificity | Primer sequence (5′-3′) | gene | size | temperature |
| SSTR1 | GGAGGAGCCGGTTGACTATT | 1140-1159 | 375 | 58° C. |
| | AAGGTAGCCTGAAAGCCTTCC | 1494-1514 | | |
| SSTR2 | AGAGCCGTACTATGACCTGA | 184-203 | 627 | 59° C. |
| | AGCCCACTCGGATTCCAGAG | 793-812 | | |
| SSTR3 | GAGCACCTGCCACATGCAGT | 661-681 | 316 | 62° C. |
| | CCCAAAGAAGGCAGGCTCCT | 938-957 | | |
| SSTR4 | TCCCTTATCCTCAGCTATGC | 948-968 | 283 | 60° C. |
| | CTCAGAAGGTGGTGGTCCTG | 1211-1251 | | |
| SSTR5 | TCTTCTCTTGCAGAGCCTGA | 11-30 | 437 | 63° C. |
| | TGACTGTCAGGCAGAAGACA | 428-447 | | |

SSTR-1, -2, -3, -4, and -5 primer pairs were designed to hybridize to unique regions of the receptors. The PCR reactions for SSTRs 1-5 were carried out using 2(1 of cDNA in 25 (1 total 5 volume of PCT buffer (67 mM Tris pH 9.01, 1.5 mM MgSO4, 166 mM AmSO4, and 10 mM (mercaptoethanol) containing 1 mM MgCl2 (5 mM MgCl2 for SSTR5), 0.2 mM dNTPs (Pharmacia), 5% DSMO (SSTR5 only) and 100 ng of 5′ and 3′ primer. Taq polymerase (1.25 U, Gibco BRL). The amplification reaction was carried out in a RoboCycler Gradient 96 (Stratagene, La Jolla, Calif.) for 35 cycles. Each cycle consisted of denaturation for 45 sec at 94° C., annealing for 10 45 sec at the relevant temperature (see Table 2), and an extension for 45 sec at 72° C. A final extension step at 72° C. for 5 min terminated the amplification. The PCR products were separated by electrophoresis through a 1% agarose gel. The DNA was visualized and photographed using the Eagle Eye II Video System (Stratagene). The DNA fragments obtained using primers for SSTR 1, 2 and 5 were isolated from the gels and ligated into pGEM-T (Stratagene, La Jolla, Calif.). DNA sequencing of the sub-clone was performed using the dideoxynucloetide chain-termination procedure with T7 sequenase (Pharmacia Biotech Inc.). The DNA fragments obtained using primers for SSTR3, and 4 were eluted from the agarose gel and diagnostic restriction digest analysis performed to confirm that the PCR products were SSTR-3 and -4.

For detection of vWF in endothelial cells, oligonucleotide primers with the sequence: 5° CCCACCCTTTGATGAACACA3′ for the forward primer and 5° CCTCACTTGCTGCACTTCCT3′ for the reverse primer were used in PCR reactions to detect von Willebrand's factor (vWF) cDNA. The PCR reaction was performed in PCR buffer (20 mM Tris-HCl (pH8.4),50 mM KCl) containing 2.0 mM MgCl2, 0.2 mM dNTPs, (Pharmacia), 5% DSMO, and 100 ng of 5′ and 3′ primer with the addition of Taq polymerase (1.25 U, Gibco BRL). The 35 PCR cycles were performed as described above with an annealing temperature of 60° C. The PCR products were separated and visualized as above. The DNA fragment was isolated from the gel and diagnostic restriction digest analysis was performed to confirm the PCR product was VWF.

### EXAMPLE 3: Effect of an SSTR1 Selective Ligand on Human Endothelial Cells

It has been demonstrated that SS acting through SSTR1 regulates intracellular pH (Barber et al., 1989, J. Biol. Chem. 264:21038) and that intracellular pH in turn regulates actin stress fiber production (Tominaga et al., 1998, Mol. Biol. Cell. 9:2287). The present Example illustrates the common effects of SS14 and an SSTR1 selective ligand agonist on actin bundling in endothelial cells, using fluorescently labelled phalloidin to localise actin.

To assay the effect of SS14 on endothelial cells, ECV304 cells were washed to remove growth medium and fresh medium (lacking serum) added (1 ml/well). The cells were cooled to 4° C. for 15 minutes to concentrate SSTRs at the plasma membrane prior to the addition of SS14 (10 nM, Peninsula Laboratories; Belmont, Calif.) to test wells while control wells received a similar volume of medium only. The cells were subsequently incubated at 37° C. for 30 min, fixed in 4% PFA for 5 min and washed in PBS. The actin cytoskeleton was visualized by incubating the cells with ALEXA-488 conjugated phalloidin (1:50, Molecular Probes Inc., Eugene, Oreg.) for 15 min at room temperature. Cells were screened using a Zeiss Axiophot microscope as previously described. Similar protocols were used to evaluate the effects SSTR1 selective ligands on endothelial cells.

In control ECV304 cells abundant stress fibres stretching the entire length of the cell and few lamellipodia were observed. The SS14-treated ECV304 cells showed a loss of long stress fibers and the remaining fibers were short and lacked directional organization. In addition, there was an increase in the number and size of lamellipodia at the plasma membrane. In addition to these morphological changes, SS14 was shown to inhibit the Na/H exchanger on ECV304 cells, as determined by intracellular pH imaging This indicates that monitoring changes to the actin cytoskeleton or intracellular pH are rapid and simple methods to follow activation of SS receptors on endothelial cells. In some embodiments, this assay may be used to screen for SSTR1 or SSTR4 selective ligands.

Treatment of ECV304 or HUVEC cells with the SSTR1 '499 agonist produced results similar to treatment of the cells with SS14. The result of SSTR1 '499 treatment was a decrease in stress fibres and an increase in lamellipodia formation. Treatment of ECV304 or HUVEC cells with a SSTR2 selective agonist, DC32-87 (Raynor et al., 1993, Mol. Pharmacol 43(6):838) had no effect on the endothelial cells.

### EXAMPLE 4: SSTRs in Human Endothelial Tissues v. Animal Tissues

In humans, the presence of mRNA for SSTR1, SSTR2 and SSTR4 (but not SSTR3 or SSTR5) was detected by RT-PCR in normal aorta, normal internal mammary artery, normal saphenous vein, and athlerosclerotic popliteal arteries. In all normal endothelial tissues, SSTR1 was expressed and was the most abundant of the receptor sub-types. The expression of SSTR2 and SSTR3 was more variable, with some individuals lacking expression of one of the two sub-types. In normal tissues, the abundance of the mRNA was lower for SSTR2 and SSTR3 compared to SSTR1.

Human artery samples (100-400 mg) were collected from bypass procedures, amputations or from human donors for organ transplantation in association with Pacific Organ Retrieval and Transplant Society with ethical permission from the Ethical Committee on Human Experimentation at the University of British Columbia. Normal veins N=6 (greater saphenous and arm), arteries N=5 (aorta and internal mammary) and diseased atherosclerotic or aneurysmal arteries N=3 were collected. The normal tissues used to obtain these results were as follows: 2 normal aortic samples, one from a 42-year-old woman and the second from a 19-year-old male; 3 internal mammary arteries and 3 saphenous veins from male patients ranging from 69-74 years of age. In athlerosclerotic popliteal arteries, SSTR1 was also the predominant receptor with variable levels of SSTR2 and SSTR4, again there was no evidence for the presence of SSTR3 or SSTR5. The 3 popliteal arteries were collected from male patients of 68, 72 and 73 years of age.

The vascular tissues analyzed herein include both endothelial and non-endothelial cells. In particular, non-endothelial smooth muscle cells form a substantial component of the vasculature. In a primary cell preparation of aortic smooth muscle cells, mRNAs for SSTR1, SSTR2 and SSTR4 were detected. In these aortic cell cultures, vWF mRNA was also detected, and vWF immunostaining (<10% of cells) was detected, indicating that the cultures included some endothelial cells.

Taken together with the results of the analysis of mRNA expression in human endothelial cells (Example 2 above), the results reported in this Example suggest that the SSTR2 mRNA detected in human vascular tissues originates with the non-endothelial cells in the tissues, while the SSTR1 and SSTR4 mRNA originates with the endothelial cells.

Immunocytochemistry was used to confirm that endothelial cells in situ expressed SSTR1. In normal and diseased blood vessels endothelial cells were immunostained by SSTR1 but not SSTR2 antibodies. Von Willebrand's Factor-immunoreactivity (IR) was limited to endothelial cells in normal and diseased vessels. For immunocytochemistry, a small portion from each vessel sample was fixed in 4% paraformaldehyde ((PFA) for 1 h and 10(m cryostat sections mounted on glass slides and cultured cells fixed for 10 min in PFA were used for immunocytochemistry. Rabbit antisera to human SSTR-1 (1: 100) and SSTR-2 (1:100) (CURE/Gastroenteric Biology Center Antibody/RIA Core, NIH grant DK 41301) and VWF (Sigma; 1:1000) were incubated on sections or whole cells at 4° C. overnight. After washing in PBS to remove excess antibodies the bound antibodies were localized using Cy3 conjugated donkey anti-rabbit IgG (Jackson ImmunoResearch Laboratories Inc., West Grove, Pa.) at 1:1000 for 1 h at room temperature. Slides were screened using a Zeiss Axiophot microscope equipped with epifluorescence. Representative sections were digitized using a Biorad MRC 600 confocal laser scanning microscope equipped with a krypton argon laser. The resultant image stacks were converted to maximum intensity projections using NIH image (share ware) and the final images produced using Adobe Photoshop.

The results of assays of SSTRs in tissue from animal models may be contrasted with the foregoing results from human tissues (see for a background example: Chen et al., 1997, J. Invest. Surg. 10:17). In control samples of rodent iliac arteries no detectable immunoreactivity was observed to antisera specific for SSTR-1, 2 and 3. However, after injury, SSTR-2 immunoreactivity was observed on the surface of the endothelial cells re-populating the injured site. The identity of the SSTR-2 immunoreactive cells and endothelial cells was confirmed by double staining with a monoclonal antibody to vWF. This immunocytochemical result indicates that SSTR-2 is the active SS receptor in the rat model of arterial injury. This was confirmed with RT-PCR using primers specific for the 5 known SSTRs. The results demonstrated that normal rat arteries expressed low levels of SSTR2 and SSTR3, but not SSTR1, SSTR4 or SSTR5. A competitive PCR protocol was used to compare the levels of SSTR2 mRNA in control and injured vessels. The results using this protocol demonstrated a clear increase in expression levels of the SSTR2 receptor 7 days after balloon injury of the rat iliac arteries. Subsequent experiments demonstrated that this increase was maintained for up to 2 months after injury. These animal model results are consistent with the ability of angiopeptin to inhibit intimal hyperplasia in rats, and hence the ability of SSTR1 and SSTR4 selective agonists to inhibit intimal hyperplasia in humans.

### EXAMPLE 5: Therapeutic Formulations

In one aspect, the invention provides a variety of therapeutic uses for SS ligands. In various embodiments, SSTR1 and SSTR4 selective ligands may be used therapeutically in formulations or medicaments for the treatment of human endothelial-cell-mediated proliferative diseases, such as pathological angiogenesis and intimal hyperplasia, including cancers susceptible to SSTR1 and SSTR4 selective ligands (such as susceptible solid tumors). The invention provides corresponding methods of medical treatment, in which a therapeutic dose of a SS ligand is administered in a pharmacologically acceptable formulation. Accordingly, the invention also provides therapeutic compositions comprising a SS ligand and a pharmacologically acceptable excipient or carrier. The therapeutic composition may be soluble in an aqueous solution at a physiologically acceptable pH. In one aspect of the invention, SSTR1 and/or SSTR4 selective ligands may be administered using a perforated balloon catheter, as disclosed in International Patent Publication WO 93/08866 of May 13, 1993, which is hereby incorporated by reference.

The invention provides pharmaceutical compositions (medicaments) containing (comprising) SS ligands. In one embodiment, such compositions include a SS ligand compound in a therapeutically or prophylactically effective amount sufficient to alter, and preferably inhibit, production of gamma interferon, and a pharmaceutically acceptable carrier. In another embodiment, the composition includes a SS ligand compound in a therapeutically or prophylactically effective amount sufficient to inhibit angiogenesis, and a pharmaceutically acceptable carrier.

The SSTR1 and SSTR4 selective ligands may be used in combination with other compositions and procedures for the treatment of diseases. For example, a tumor may be treated conventionally with photodynamic therapy, surgery, radiation or chemotherapy combined with a SSTR1 or SSTR4 selective ligand, and then a SSTR1 or SSTR4 selective ligand may be subsequently administered to the patient to extend the dormancy of micrometastases and to stabilize and inhibit the growth of any residual primary tumor.

In another aspect an SSTR ligand may be used as a vehicle for transporting a medicament, such as a photosensitizer or other chemotherapeutic agent, to specific receptor so that the medicament may be localized on the surface of target cells or internalized by the target cells. For instance, a photosensitizer may covalently be linked to a ligand so that the photosensitizer is available for photodynamic therapy (PDT) and the ligand is available to interact with the specific receptor, using for example methods such as those disclosed in, but not limited to, U.S. Pat. No. 5,171,749 issued Dec. 15, 1992.

An effective amount of a ligand compound of the invention may include a therapeutically effective amount or a prophylactically effective amount of the compound. A “therapeutically effective amount” generally refers to an amount effective, at dosages and for periods of time necessary, to achieve the desired therapeutic result, such as reduction or reversal of angiogenesis in the case of cancers, or reduction or inhibition intimal hyperplasia. A therapeutically effective amount of SS ligand may vary according to factors such as the disease state, age, sex, and weight of the individual, and the ability of the SS ligand to elicit a desired response in the individual. Dosage regimens may be adjusted to provide the optimum therapeutic response. A therapeutically effective amount is also one in which any toxic or detrimental effects of the SS ligand are outweighed by the therapeutically beneficial effects. A “prophylactically effective amount” refers to an amount effective, at dosages and for periods of time necessary, to achieve the desired prophylactic result, such as preventing or inhibiting the rate of metastasis of a tumour or the onset of intimal hyperplasia. A prophylactically effective amount can be determined as described above for the therapeutically effective amount. Typically, since a prophylactic dose is used in subjects prior to or at an earlier stage of disease, the prophylactically effective amount will be less than the therapeutically effective amount.

In particular embodiments, a preferred range for therapeutically or prophylactically effective amounts of a SSTR1 or SSTR4 selective ligand may be 0.1 nM-0.1M, 0.1 nM-0.05M, 0.05 nM-15 μM or 0.01 nM-10 μM. Alternatively, total daily dose may range from about 0.001 to about 1 mg/kg of patients body mass. Dosage values may vary with the severity of the condition to be alleviated. It is to be further understood that for any particular subject, specific dosage regimens should be adjusted over time according to the individual need and the professional judgement of the person administering or supervising the administration of the compositions, and that dosage ranges set forth herein are exemplary only and are not intended to limit the scope or practice of the methods of the invention.

The amount of active SSTR selective ligand in a therapeutic composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the SS ligands can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound (e.g.SS ligand) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, a SS ligand may be formulated with one or more additional compounds that enhance the solubility of the SS ligand.

A further form of administration is to the eye. An SSTR1 or SSTR4 selective ligand may be delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material. Alternatively, the compounds of the invention may be injected directly into the vitreous and aqueous humour. In a further alternative, the compounds may be administered systemically, such as by intravenous infusion or injection, for treatment of the eye. In some embodiments, anti-angiogenic treatment with SSTR1 or SSTR4 ligands may be undertaken following photodynamic therapy (such as is described in U.S. Pat. No. 5,798,349 issued Aug. 25, 1998, incorporated herein by reference).

In accordance with another aspect of the invention, therapeutic compositions of the present invention, comprising SSTR1 or SSTR4 selective ligands, may be provided in containers having labels that provide instructions for use of, or to indicate the contents as, SSTR1 or SSTR4 selective ligands to treat endothelial-cell-mediated proliferative diseases.

### EXAMPLE 6

In this example, the expression and cellular localization of SSTR1 in the vasculature of the eye is demonstrated. A consistent finding is that SSTR-1 is expressed on endothelial cells in normal eyes and eyes with macular degeneration.

Normal human retina sections (N=2) and normal human sclera sections (N=3) were immunostained with an antibody to SSTR-1, SSTR-2, and vWF. SSTR-1-immunoreactivity (IR) was observed on endothelial cells in the blood vessels of the macula area. There was no SSTR-2-IR in the endothelial cells of the blood vessels. VWF-IR was located in the endothelial cells of the blood vessels.

An eye sample from a patient with macular degeneration was immunostained with antisera to SSTR-1, -2, and the NK1 (substance P) receptor. The results were similar to normal eye tissue, SSTR-1 was found in the endothelial cells, SSTR-2 was absent, substance P receptor-IR was localized to the blood vessel. In subretinal neovascular ‘membrane’ sections (N=5) that were positively stained, SSTR-1-IR was frequently co-localized with vWF-IR. SSTR-2-IR was not observed.

All references cited herein are hereby incorporated by reference in their entireties, whether previously specifically incorporated or not. As used herein, the terms “a”, “an”, and “any” are each intended to include both the singular and plural forms.

What is claimed is:

1. A method for inhibiting angiogenesis in a human patient in need of such inhibition, comprising administering to said patient an effective amount of a somatostatin receptor agonist ligand that binds SSTR1.

2. A method for inhibiting angiogenesis in a human patient in need of such inhibition, comprising administering to said patient an effective amount of a somatostatin receptor agonist ligand that binds SSTR4.

3. A method for treating age-related macular degeneration in a human patient in need of such treatment, comprising administering to said patient an effective amount of a somatostatin receptor agonist ligand that binds SSTR1.

4. A method for treating age-related macular degeneration in a human patient in need of such treatment, comprising administering to said patient an effective amount of a somatostatin receptor agonist ligand that binds SSTR4.

5. A method for inhibiting the angiogenic activity of human endothelial cells in a patient in need of such inhibition comprising administering to said patient an effective amount of a somatostatin receptor agonist ligand that binds SSTR1.

6. A method for inhibiting the angiogenic activity of human endothelial cells in a patient in need of such inhibition comprising administering to said patient an effective amount of a somatostatin receptor ligand that binds with greater affinity to SSTR4 than to any other somatostatin receptor.

7. The method of claim 3 wherein the somatostatin receptor ligand is administered after the patient has undergone photodynamic therapy.

8. The method of claim 4 wherein the somatostatin receptor ligand is administered after the patient has undergone photodynamic therapy.

9. A method for treating cancer by inhibiting solid tumour angiogenesis which comprises administering to a human patient in need of such treatment an effective amount of a somatostatin receptor ligand that binds SSTR1.

10. A method for treating cancer by inhibiting solid tumour angiogenesis which comprises administering to a human patient in need of such treatment an effective amount of a somatostatin receptor ligand that binds SSTR4.

11. The method of claim 1 wherein the somatostatin receptor ligand is SS14 or des-AA^{1,2,5} [DTrp⁸,IAmp⁹]SS.

12. The method of claim 2 wherein the somatostatin receptor ligand is SS14.

13. The method of claim 3 wherein the somatostatin receptor ligand is SS14 or des-A,A^{1,2,5} [DTrp⁸,IAamp⁹]SS.

14. The method of claim 4 wherein the somatostatin receptor ligand is SS14.

15. The method of claim 5 wherein the somatostatin receptor ligand is SS14 or des-AA^{1,2,5} [DTrp⁸ ,IAamp⁹]SS.

16. The method of claim 6 wherein the somatostatin receptor ligand is SS14.

17. The method of claim 9 wherein the somatostatin receptor ligand is SS14 or des-AA^{1,2,5} [DTrp⁸,IAamp⁹]SS.

18. The method of claim 10 wherein the somatostatin receptor ligand is SS 14.

19. The method of claim 1, wherein the patient is suffering from a disease selected from the group consisting of: neovascularization of the choroid and retina, iris and cornea, retinopathy of prematurity, corneal graft rejection, retrolental fibroplasia, rubeosis, hypoxia, angiogenesis in the eye associated with infection, angiogenic aspects of skin diseases, psoriasis, hemagiomas, capillary proliferation within atherosclerotic plaques, Osler-Webber Syndrome, myocardial angiogenesis, atherosclerotic plaque neovascularization, telangiectasia, hemophiliac joints', angiofibroma, wound granulation, intestinal adhesions, Crohn's disease, scleroderma, hypertrophic scars, keloids, cat scratch disease and ulcers.

20. The method of claim 1, wherein the somatostatin receptor ligand is des-AA^{1,2,5}[DTrp⁸ ,IAamp⁹]SS.
